(19) United States
(12) Patent Application Publication
(10) Pub. No.: US 2017/0224254 A1
(43) Pub. Date: Aug. 10, 2017

(54) ANALYZING SYSTEM AND ANALYZING METHOD FOR EVALUATING CALORIE CONSUMPTION BY DETECTING THE INTENSITY OF WIRELESS SIGNAL

(71) Applicant: INDUSTRIAL TECHNOLOGY RESEARCH INSTITUTE, Hsinchu (TW)

(72) Inventors: Yi-Ta CHUANG, New Taipei City (TW); Chia-Liang KE, Changhua City (TW); Yu-Chee TSENG, Hsinchu City (TW)

(73) Assignee: INDUSTRIAL TECHNOLOGY RESEARCH INSTITUTE, Hsinchu (TW)

(21) Appl. No.: 15/183,847

(22) Filed: Jun. 16, 2016

(30) Foreign Application Priority Data
Feb. 4, 2016 (TW) 105103768

(51) Int. Cl. A61B 5/11; H04W 4/02; A61B 5/00; H04B 17/318

(52) U.S. Cl. CPC A61B 5/1118; H04B 17/318; H04W 4/027; A61B 5/7278

(57) ABSTRACT

An analyzing system and a method for evaluating calorie consumption by detecting the intensity of wireless signal are provided. The analyzing system includes a wireless receiving device, a processor and a calorie consumption computing device. The wireless receiving device receives a first wireless signal emitted from a first wireless transmitting device. The wireless receiving device detects a first intensity of the first wireless signal at a first time, and detects a second intensity of the first wireless signal at a second time. The processor obtains a motion information corresponding from the first time to the second time according to the first intensity of the first wireless signal and the second intensity of the first wireless signal. The calorie consumption computing device generates a calorie consumption according to the motion information.

This application claims the benefit of Taiwan application Serial No. 105103768, filed Feb. 4, 2016, the disclosure of which is incorporated by reference herein in its entirety.

### TECHNICAL FIELD

The disclosure relates in general to an analyzing system and an analyzing method for evaluating calorie consumption by detecting the intensity of wireless signal.

### BACKGROUND

Conventional way of calculating calorie consumption is estimated by detecting motion information, such as motion step count, motion time, and motion type, using a motion monitoring device. The conventional motion monitoring device normally goes with an inertial measurement unit, such as an accelerometer speedometer or a gyroscope, to detect the user's instant motion to obtain information such as motion step count, or goes with the global positioning system (GPS) technology to obtain the user's motion region and rate of movement. However, the motion monitoring device normally has to be worn by the user when making a motion. And the power capacity of wearable devices is still limited. When the inertial measurement unit is used together with the GPS technology to detect the user's motion information, a large amount of power consumption is required, and this will make long duration motion recording difficult to achieve. Furthermore, the GPS technology may easily generate errors if the movement of an object is within a short distance. Therefore, it has become a prominent task for the industries to provide a new device and a new method for monitoring motion amount and calculating calorie consumption.

### SUMMARY

According to one embodiment, an analyzing system for evaluating calorie consumption by detecting the intensity of wireless signal is provided. The analyzing system includes a wireless receiving device, a processor and a calorie consumption computing device. The wireless receiving device receives a first wireless signal emitted from a first wireless transmitting device. The wireless receiving device detects a first intensity of the first wireless signal at a first time and detects a second intensity of the first wireless signal at a second time. The processor obtains a motion information corresponding from the first time to the second time according to the first intensity of the first wireless signal and the second intensity of the first wireless signal. The calorie consumption computing device generates a calorie consumption according to the motion information.

According to another embodiment, an analyzing method for evaluating calorie consumption by detecting the intensity of wireless signal is provided. The analyzing method includes following steps. Firstly, a first wireless signal emitted from a first wireless transmitting device is received. Next, a first intensity of the first wireless signal is detected at a first time. Then, a second intensity of the first wireless signal is detected at a second time. After that, a motion information corresponding from the first time to the second time is obtained according to the first intensity of the first wireless signal and the second intensity of the first wireless signal. Finally, a calorie consumption is generated according to the motion information.

The above and other aspects of the invention will become better understood with regard to the following detailed description of the preferred but non-limiting embodiment (s). The following description is made with reference to the accompanying drawings.

### BRIEF DESCRIPTION OF THE DRAWINGS

FIG. 1 shows a flowchart of an analyzing method for evaluating calorie consumption by detecting the intensity of wireless signal according to an embodiment of the disclosure.

FIG. 2 shows a block diagram of an analyzing system for evaluating calorie consumption by detecting the intensity of wireless signal according to an embodiment of the disclosure.

FIG. 3 shows a schematic diagram of an analyzing method for evaluating calorie consumption by detecting the intensity of wireless signal according to an embodiment of the disclosure.

FIG. 4 shows a schematic diagram of a user's motion information within a time period according to the disclosure.

FIG. 5 shows a block diagram of an analyzing system for evaluating calorie consumption by detecting the intensity of wireless signal according to another embodiment of the disclosure.

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

### DETAILED DESCRIPTION

The disclosure is for obtaining motion information by detecting the intensity of the received wireless signal, calculating calorie consumption according to the obtained motion information, and recording the user's motion information for analyzing the user's motion habit such as motion region, motion time or motion step count.

The disclosure is for creating a conversion model of the wireless signal intensity state and the motion step count and further adjusting the conversion model according to different user's situations, such that the calorie consumption can be calculated more accurately.

According to the disclosure, a calorie consumption is evaluated by detecting the intensity of wireless signal, a motion information corresponding from the first time to the second time is obtained by detecting the intensity of the ambient wireless signal at the first time and the intensity of the ambient wireless signal at the second time, and a corresponding calorie consumption can be calculated according to the obtained motion information. In comparison to the method using GPS technology or the method detecting acceleration using an inertia measurement unit, the method of the disclosure detects the motion information according to the received wireless signal, hence consuming less power. Unlike the prior art which needs to continuously detects instant acceleration at each motion, the disclosure only needs to detect the intensity of the wireless signal every period of time, hence largely reducing power consumption.

Moreover, the disclosure further creates a corresponding conversion model with respect to the user's personal motion habit or particular motion region, such that the calorie consumption can be more accurately calculated.

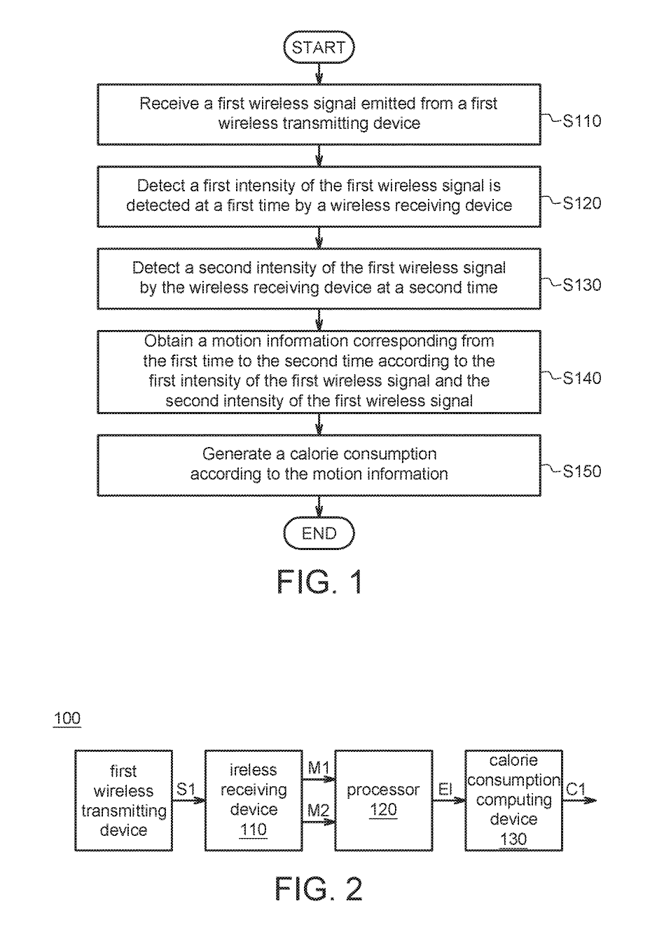

FIG. 1 shows a flowchart of an analyzing method for evaluating calorie consumption by detecting the intensity of wireless signal according to an embodiment of the disclosure. The analyzing method includes following steps. Firstly, in step S110, a first wireless signal emitted from a first wireless transmitting device is received. Next, in step S120, a first intensity of the first wireless signal is detected by a wireless receiving device at a first time. Then, in step S130, a second intensity of the first wireless signal is detected by the wireless receiving device at a second time. After step S130, in step S140, a motion information corresponding from the first time to the second time is obtained according to the first intensity of the first wireless signal and the second intensity of the first wireless signal. Finally, in step S150, a calorie consumption is generated according to the motion information.

To put it in greater details, the analyzing method can be used in the analyzing system of FIG. 2. FIG. 2 shows a block diagram of an analyzing system for evaluating calorie consumption by detecting the intensity of wireless signal according to an embodiment of the disclosure. The analyzing system for evaluating calorie consumption by detecting the intensity of wireless signal 100 includes a wireless receiving device 110, a processor 120 and a calorie consumption computing device 130. The wireless receiving device 110 receives a first wireless signal S1 emitted from a first wireless transmitting device. The wireless receiving device 110 detects a first intensity M1 of the first wireless signal S1 at a first time and detects a second intensity M2 of the first wireless signal S1 at a second time. The processor 120 obtains a motion information EI corresponding from the first time to the second time according to the first intensity M1 of the first wireless signal S1 and the second intensity M2 of the first wireless signal S1. The calorie consumption computing device 130 generates a calorie consumption C1 according to the motion information EI.

In an embodiment, the wireless receiving device 110 and the processor 120 may include in a wearable electronic device such as a mobile phone, a smart watch, or a pair of smart glasses. The wireless receiving device 110 may receive the first wireless signal S1 emitted from the first wireless transmitting device. Furthermore, as the user moves, the intensity of the first wireless signal S1 received by the wireless receiving device 110 will change. Any person ordinarily skilled in the art will understand that the intensity of a wireless signal is inversely proportional to the square of distance. Therefore, the user's moving distance can be obtained according to the intensity variation of wireless signal before and after the movement of the user. In the disclosure, the user's moving distance can be converted into motion step count. The user's motion region can be obtained according to the wireless signals with stronger intensities among the received wireless signals. Furthermore, the user's motion time is recorded at the same time when the wireless signal is received. Therefore, the processor 120 obtains a motion information EI corresponding from the first time to the second time according to the first intensity M1 of the first wireless signal S1 detected at the first time of the first wireless signal S1 detected at the first time and the second intensity M2 of the first wireless signal S1 detected at the second time. The motion information EI includes a motion time, a motion region and a motion step count. After that, the calorie consumption computing device 130 generates a calorie consumption C1 according to the motion information. The processor can be realized by a microprocessor, a central processing unit (CPU), or various application circuits or software.

In an embodiment, the calorie consumption computing device 130 may include in the same wearable electronic device. In another embodiment, the calorie consumption computing device 130 may include in another device, such as a remote server. And the processor 120 may transmit the motion information to the calorie consumption computing device 130 disposed in the remote server. In the embodiment, the first wireless transmitting device and the wireless receiving device 110 may employ wireless transmission such as but not limited to Bluetooth, Wi-Fi, and ZigBee.

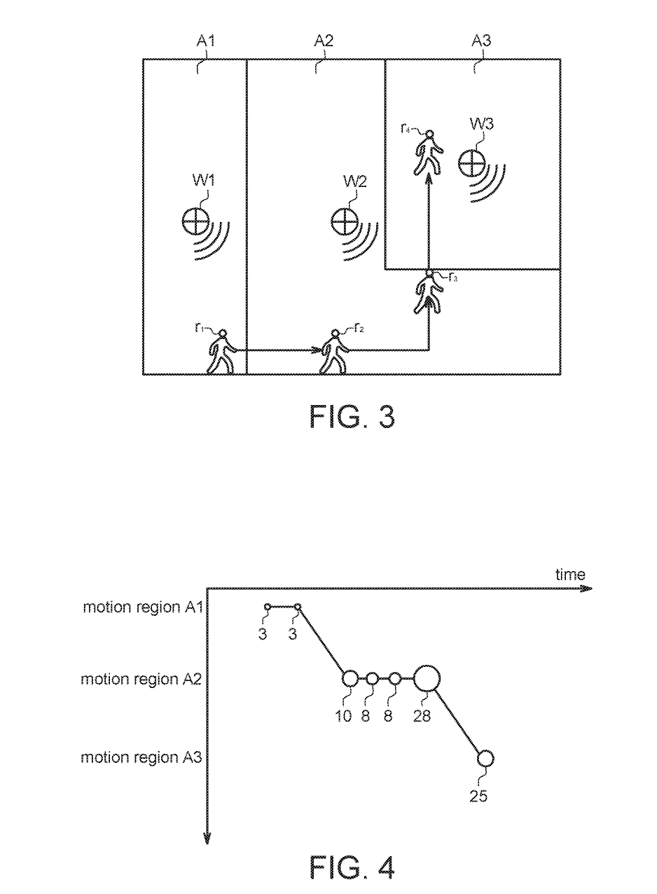

The method of the disclosure for evaluating calorie consumption by detecting the intensity of wireless signal is exemplified below with accompanying drawings. Referring to FIG. 3, a schematic diagram of an analyzing method for evaluating calorie consumption by detecting the intensity of wireless signal according to an embodiment of the disclosure is shown. In the example, multiple wireless transmitting devices may be disposed within the user's motion region. As indicated in FIG. 3, the user's motion region can be divided into three motion regions A1, A2 and A3 within which three wireless transmitting devices W1, W2, and W3 are disposed respectively. The user is located at position r₁ of the motion region A1 at a first time, and afterwards, the user moves to position r₂ of the motion region A2 at a second time, and then moves to position r₃ of the motion region A2 at a third time, and continues to move to position r₄ of the motion region A3 at a fourth time.

In an embodiment, the wireless receiving device 110 at position r1 may receive the intensities of three wireless signals from the first wireless transmitting device W1, the second wireless transmitting device W2 and the third wireless transmitting device W3 at the first time, and the wireless receiving device 110 at position r2 may receive the intensities of three wireless signals from the first wireless transmitting device W1, the second wireless transmitting device W2 and the third wireless transmitting device W3 at the second time. The processor 120 obtains a motion information EI corresponding from the first time to the second time according to the intensities of the wireless signals received at the first time and the intensities of the wireless signals received at the second time. As disclosed above, the user's moving distance may be obtained according to the intensity variation of the wireless signals emitted from the first wireless transmitting device W1, the second wireless transmitting device W2 and the third wireless transmitting device W3 before and after the movement of the user. In the disclosure, the user's moving distance may be converted into motion step count. The user's motion region may be obtained according to the wireless signals with stronger intensities among the received wireless signals. The user's motion time is recorded at the same time when the wireless signals are received. Similarly, the user's moving distance (motion step count), motion region, and motion time may be obtained according to the intensity variation of wireless signals before and after the movement of the user. Similarly, the wireless receiving device 110 at position r2 may receive the intensities of three wireless signals from the first, the second, and the third wireless transmitting devices W1, W2 and W3 at the second time, and the wireless receiving device 110 at position r3 can receive the intensities of three wireless signals from the wireless transmitting devices W1, W2 and W3 at the third time. The processor 120 may obtains a motion information EI corresponding from the second time to the third time according to the intensities of the wireless signals received at the second time and the intensities of the wireless signals received at the third time.

In one embodiment, the time interval from the first time to the second time may be the same as the time interval from the second time to the third time. However, the disclosure is not limited thereto. For example, the time interval from the first time to the second time may be different from the time interval from the second time to the third time. That is, any person ordinarily skilled in the art may determine a fixed time interval at which the intensity of wireless signal is detected.

In one embodiment, the processor 120 converts detected intensities of wireless signals into a corresponding motion step count according to a conversion model. The conversion model, such as a polynomial model or a lookup table, is a corresponding relationship between the wireless signal intensity state and the motion step count, and may be built in the analyzing system 100 and adjusted or updated as the user or the environment varies. The conversion model may be obtained from a machine learning technique, such as a support vector machine (SVM). The learning steps are as follows. In an environment, n wireless transmitting devices are disposed, and the wireless receiving device worn by the user may receive wireless signals from the n wireless transmitting devices to obtain an n-dimensional signal intensity vector. During the motion process, if the motion step count from the start point to the destination point and the detected number of signal vectors may be recorded for each walk, then each motion information may be denoted as <rₛ, r_{d}, p, step>, wherein rₛ represents the signal intensity vector detected at the starting point; r_{d} represents the signal intensity vector detected at the destination point; p represents the number of signal intensity vectors detected during the process; step represents the motion step count between the starting point rₛ and the destination point r_{d}. During the learning process, several items of motion information and corresponding intensity vectors of wireless signals may be collected and further inputted to a support vector machine to generate a conversion model. Also, detailed method and mathematical model for training support vector machine are disclosed in following reference: R.-E. Fan, P.-H. Chen, and C.-J. Lin. “Working set selection using the second order information for training SVM.” Journal of Machine Learning Research 6, 1889-1918, 2005.

For example, Table 1 illustrates the motion step counts corresponding to the intensities of wireless signals detected by the wireless receiving device 110. The motion step counts are obtained by the processor 120 with reference to a conversion model.

TABLE 1

| | Intensity of wireless | Intensity of wireless | |
|---|---|---|---|
| | signal corresponding | signal corresponding | |
| | to the position before | to the position after | Motion Step |
| Motion | motion (dB) | motion (dB) | count |
| From r₁ to r₂ | (−60, −80, −150) | (−70, −65, −100) | 3 |
| From r₂ to r₃ | (−70, −65, −100) | (−100, −60, −70) | 2 |
| From r₃ to r₄ | (100, −60, −70) | (−150, −75, −50) | 4 |

As indicated in the first row of Table 1, the intensities of the three wireless signals emitted from the first wireless transmitting device W1, the second wireless transmitting device W2 and the third wireless transmitting device W3 and received by the wireless receiving device 110 at the first time when the wireless receiving device 110 is at position r₁ are −60 (dB), −80 (dB) and −150 (dB), respectively. The intensities of the three wireless signals emitted from the wireless transmitting devices W1, W2 and W3 and received by the wireless receiving device 110 at the second time when the wireless receiving device 110 is at position r₂ are −70 (dB), −65 (dB) and −100 (dB), respectively. Thus, the processor 120, according to the intensities of the wireless signals (−60, −80, −150) detected at the first time and the intensities of the wireless signals (−70, −65, −100) detected at the second time, may determine that the motion step count corresponding from the first time to the second time is 3 steps. Similarly, as indicated in the second row of Table 1, the intensities of the three wireless signals emitted from the wireless transmitting devices W1, W2 and W3 and received by the wireless receiving device 110 at the second time when the wireless receiving device 110 is at position r₂ are (−70, −65, −100), respectively. The intensities of the three wireless signals emitted from the wireless transmitting devices W1, W2 and W3 and received by the wireless receiving device 110 at the third time when the wireless receiving device 110 is at position r₃ are (−100, −60, −70), respectively. Thus, the processor 120 may determine that the motion step count corresponding from the second time to the third time is 2 steps. As indicated in the third row of Table 1, the intensities of the three wireless signals emitted from the wireless transmitting devices W1, W2 and W3 and received by the wireless receiving device 110 at the third time when the wireless receiving device 110 is at position r3 are (−100, −60, −70), respectively. The intensities of the three wireless signals emitted from the wireless transmitting devices W1, W2 and W3 and received by the wireless receiving device 110 at the fourth time when the wireless receiving device 110 is at position r4 are (−150, −75, −50), respectively. Thus, the processor 120 may determine that the motion step count corresponding from the third time to the fourth time is 4 steps. In one embodiment, if the wireless signal emitted from the wireless transmitting device cannot be detected, then the intensity of wireless signal is set as −150 (dB).

After the motion step count is obtained, the calorie consumption computing device 130 may calculate the corresponding calorie consumption C1. For example, the calorie consumption C1 (kcal) may be calculated as: C1=weight*(steps count)*(0.78/1000)*1.036. Suppose the user weighs 80 kg. The calorie consumption C1 corresponding from the first time to the fourth time may be calculated as: C1=80*(3+2+4)*(0.78/1000)*1.036=0.58 (kcal). However, the disclosure is not limited thereto. For example, the calorie consumption computing device 130 may calculate the calorie consumption C1 corresponding from the first time to the fourth time according to the user's gender, age and pace.

In an embodiment, the processor 120 may determine the user's motion region detected at the first time according to the intensities of wireless signals emitted from the wireless transmitting device W1, the wireless transmitting device W2 and the wireless transmitting device W3 and received by the wireless receiving device 110 at the first time. For example, since the intensity of the wireless signal received from the wireless transmitting device W1 at the first time being −60 (dB) is larger than the intensity of the wireless signal received from the wireless transmitting device W2 being −80 (dB) as well as the intensity of the wireless signal received from the wireless transmitting device W3 being −150 (dB), the processor 120 may determine that the user's motion region at the first time is A1. Likewise, since the intensity of the wireless signal received from the wireless transmitting device W2 at the second time being −65 (dB) is larger than the intensity of the wireless signal received from the wireless transmitting device W1 being −70 (dB) as well as the intensity of the wireless signal received from the wireless transmitting device W3 being −100 (dB), the processor 120 may determine that the user's motion region at the second time is A2. Since the intensity of the wireless signal received from the wireless transmitting device W2 at the third time being −60 (dB) is larger than the intensity of the wireless signal received from the wireless transmitting device W1 being −100 (dB) as well as the intensity of the wireless signal received from the wireless transmitting device W3 being −70 (dB), the processor 120 may determine that the user's motion region at the third time is A2. In one embodiment, since the position r3 is close to the boundaries of the motion region A2 and the motion region A3, the processor 120 will determines the user's motion region at the third time as A3 if the intensity of wireless signal the wireless transmitting device W3 is larger than the intensity of the wireless signal received from the wireless transmitting device W2. In another embodiment, the wireless receiving device 110 further detects the average received signal strength indication (RSSI) received from the wireless transmitting device at a fixed time interval, and the user's current motion region may be obtained according to the wireless transmitting device with the strongest signal.

Therefore, the processor 120 may obtain a corresponding motion information EI according to multiple intensities of multiple wireless signals. The motion information EI may include a motion time, a motion region and a motion step count. Referring to FIG. 4, a schematic diagram of a user's motion information within a time period according to the disclosure is shown. As indicated in FIG. 4, the user's first motion information indicates that the user is within the motion region A1, and after walking for 3 steps, the user is still within the motion region A1. Then, the user walks for 3 steps to the motion region A2, and continues to walk for 10 steps within the motion region A2. Then, the user walks for 8 steps twice within the motion region A2 and then walks for 28 steps to the motion region A3. Finally, the user walks for 25 steps within the motion region A3. Thus, the analyzing system 100, according to the above motion information, may obtain the user's motion time, such as morning, noon, afternoon or evening, the duration of each motion, the motion region, the motion step count, and the calorie consumption. The analyzing system 100 may further obtain the motion amount variation of the user according to the statistics of the motion information over several days. Thus, the analyzing system 100 is convenient for the user to record and analyze his/her personal motion habit.

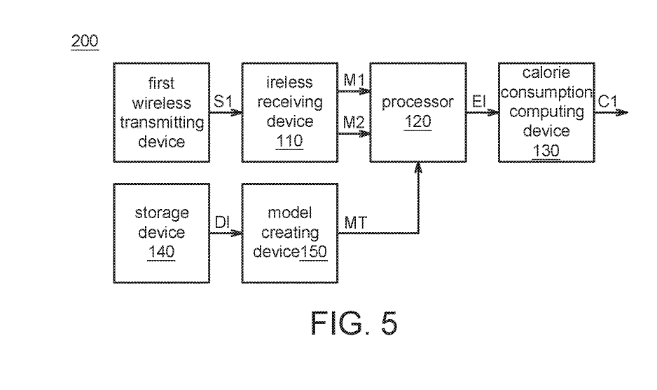

Referring to FIG. 5, a block diagram of an analyzing system for evaluating calorie consumption by detecting the intensity of wireless signal 200 according to another embodiment of the disclosure is shown. The analyzing system 200 of FIG. 5 is different from the analyzing system 100 of FIG. 2 in that the analyzing system 200 further includes a storage device 140 and a model creating device 150.

In one embodiment, the storage device 140 stores multiple motion records DI. Each motion record DI is such as the user's walk within a region over a period of time. Each motion record DI includes a wireless signal intensity state of at least one wireless signal and at least one motion step count. The user can create multiple motion records DI. Each motion record DI records the intensity of wireless signal detected by the wireless receiving device 110 before and after the user's motion and the motion step count corresponding to each of the motion records DI. In an embodiment, the motion step count corresponding to each of the motion records DI is inputted by a user. For example, in each motion record DI, the user walks for 20 steps and detects the intensity of wireless signal before and after the user's motion. Or, the user detects the intensity of wireless signal before and after the user's motion every 10 steps to collect more records. Or, the user the user detects the intensity of wireless signal before and after the user's motion every 10 seconds to collect more records.

In another embodiment, an inertial measurement unit is used to detect the motion step count corresponding to each of the motion records DI. For example, in each motion record DI, the user uses the inertial measurement unit to detect the motion step count and uses the wireless receiving device to detect the intensity of wireless signal before and after the user's motion. Or, in each motion record, the user uses the inertial measurement unit to detect the motion step count and the wireless receiving device detects the intensity variation of wireless signal in each step to collect more records.

After collecting multiple motion records DI, the model creating device 150 creates a conversion model MT according to the motion records DI. As disclosed above, there is a corresponding relationship existing between the wireless signal intensity state and the motion step count, and the corresponding relationship may be expressed as a polynomial model or a lookup table. Any person ordinarily skilled in the art may create the conversion model MT according to the motion records DI using various statistical methods, such as regression analysis, or various mathematical algorithms. Furthermore, the conversion model may be adjusted according to parameters such as the type of the wireless transmitting device and the user's pace. Thus, the processor 120 may generate a corresponding motion step count according to the conversion model MT and the intensity variation of wireless signal. For example, in the conversion model MT, the corresponding relationship between the intensity of wireless signal and the motion step count is expressed as a polynomial, and the processor 120 may substitute the first intensity M1 of the first wireless signal and the second intensity M2 of the first wireless signal to the polynomial to obtain a motion step count corresponding from the first time to the second time. After that, the calorie consumption computing device may generate a calorie consumption C1 according to the motion step count obtained in the conversion model MT.

Many analyzing systems and methods for evaluating calorie consumption by detecting the intensity of wireless signal are provided in above embodiments. A motion information corresponding from the first time to the second time may be obtained by detecting the intensity of the wireless signal from the environment at the first time and the intensity of the wireless signal from the environment at the second time. Furthermore, a corresponding calorie consumption may be calculated according to the motion information. Any person ordinarily skilled in the art will understand that the power consumption required for receiving wireless signals is lower than that required for using GPS technology or that required for detecting acceleration using an inertial measurement unit. Furthermore, in the disclosure, since there is no need to detect instant acceleration of a motion, long duration of operation can be avoided, and the motion information may be obtained by detecting the intensity variation of wireless signal within a time period. In comparison to the prior art in which the acceleration needs to be detected at the instant of each motion, power consumption may be largely reduced in the disclosure. Therefore, the analyzing system and the analyzing method of the disclosure for evaluating calorie consumption by detecting the intensity of wireless signal are capable of providing a low power-consumption device and method for detecting personal motion information and calculating corresponding calorie consumption.

Besides, the analyzing system of the disclosure may create a conversion model for the wireless signal intensity state of wireless signal and the motion step count in advance, and may further adjust the conversion model as the user varies. The analyzing system and method of the disclosure for evaluating calorie consumption by detecting the intensity of wireless signal may further analyze personal motion information for the user to conveniently record and analyze his/her personal motion habit.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. An analyzing system for evaluating calorie consumption by detecting the intensity of wireless signal, comprising:
a wireless receiving device for receiving a first wireless signal emitted from a first wireless transmitting device, wherein the wireless receiving device detects a first intensity of the first wireless signal at a first time and detects a second intensity of the first wireless signal at a second time;
a processor for obtaining a motion information corresponding from the first time to the second time according to the first intensity of the first wireless signal and the second intensity of the first wireless signal; and
a calorie consumption computing device for generating a calorie consumption according to the motion information.

2. The analyzing system according to claim 1, wherein the motion information comprises a motion time, a motion region and a motion step count.

3. The analyzing system according to claim 2, wherein the wireless receiving device further receives a second wireless signal emitted from a second wireless transmitting device, and detects a third intensity of the second wireless signal at the first time and a fourth intensity of the second wireless signal at the second time;
wherein the processor further obtains the motion step count corresponding from the first time to the second time according to the first intensity, the second intensity, the third intensity and the fourth intensity;
wherein the processor determines the motion region at the first time according to the first intensity and the third intensity and determines the motion region at the second time according to the second intensity and the fourth intensity.

4. The analyzing system according to claim 3, wherein the first wireless transmitting device is located within a first region, the second wireless transmitting device is located within a second region, the processor determines whether the motion region at the first time is within the first region or the second region according to the first intensity and the third intensity, and determines whether the motion region at the second time is within the first region or the second region according to the second intensity and the fourth intensity.

5. The analyzing system according to claim 2, further comprising:
a storage device for storing a plurality of motion records, each of the motion records comprising a wireless signal intensity state and at least one motion step count; and
a model creating device for creating a conversion model according to the motion records;
wherein the processor generates the motion step count corresponding from the first time to the second time according to the conversion model and the first intensity of the first wireless signal and the second intensity of the first wireless signal.

6. The analyzing system according to claim 5, further comprising an inertial measurement unit for detecting the motion step count of each of the motion records.

7. The analyzing system according to claim 5, wherein the motion step count of each of the motion records are inputted by a user.

8. An analyzing method for evaluating calorie consumption by detecting the intensity of wireless signal, comprising:
receiving a first wireless signal emitted from a first wireless transmitting device;
detecting a first intensity of the first wireless signal at a first time;
detecting a second intensity of the first wireless signal at a second time;
obtaining a motion information corresponding from the first time to the second time according to the first intensity of the first wireless signal and the second intensity of the first wireless signal; and
generating a calorie consumption according to the motion information.

9. The analyzing method according to claim 8, wherein the motion information comprises a motion time, a motion region and a motion step count.

10. The analyzing method according to claim 9, further comprising:
receiving a second wireless signal emitted from a second wireless transmitting device;
detecting a third intensity of the second wireless signal at the first time;
detecting a fourth intensity of the second wireless signal at the second time;
obtaining the motion step count corresponding from the first time to the second time according to the first intensity, the second intensity, the third intensity and the fourth intensity of the second wireless signal;
determining the motion region at the first time according to the first intensity and the third intensity; and
determining the motion region at the second time according to the second intensity and the fourth intensity.

11. The analyzing method according to claim 10, wherein the first wireless transmitting device is located within a first region, the second wireless transmitting device is located within a second region, and the analyzing method further comprises:
determining whether the motion region at the first time is within the first region or the second region according to the first intensity and the third intensity; and
determining whether the motion region at the second time is within the first region or the second region according to the second intensity and the fourth intensity.

12. The analyzing method according to claim 9, further comprising:
storing a plurality of motion records, each of the motion records comprising a wireless signal intensity state of the first wireless signal and at least one motion step count;
creating a conversion model according to the motion records; and
generating the motion step count corresponding from the first time to the second time according to the conversion model and the first intensity of the first wireless signal and the second intensity of the first wireless signal.

13. The analyzing method according to claim 12, further comprising:
detecting the motion step count of each of the motion records by using an inertial measurement unit.

14. The analyzing method according to claim 12, further comprising:
inputting the motion step count of each of the motion records by a user.
